(19) United States
(12) Patent Application Publication
(10) Pub. No.: US 2025/0006990 A1
(43) Pub. Date: Jan. 2, 2025

(54) ELECTROLYTE ADDITIVES FOR SOLID ELECTROLYTE INTERFACE FORMATION ON POLYTETRAFLUOROETHYLENE-CONTAINING NEGATIVE ELECTRODES AND BATTERIES INCLUDING THE SAME

(71) Applicant: GM GLOBAL TECHNOLOGY OPERATIONS LLC, Detroit, MI (US)

(72) Inventors: Dewen KONG, Shanghai (CN); Si Chen, Shanghai (CN); Ziqi Wei, Guangzhou (CN); Lidan Xing, Guangzhou (CN); Haijing Liu, Shanghai (CN)

(21) Appl. No.: 18/446,262

(22) Filed: Aug. 8, 2023

(30) Foreign Application Priority Data
Jun. 27, 2023 (CN) 202310771523.5

(51) Int. Cl. H01M 10/0567; C07C 311/48; H01M 4/04; H01M 4/133; H01M 4/62; H01M 10/42

(52) U.S. Cl. CPC H01M 10/0567; C07C 311/48; H01M 4/0447; H01M 4/133; H01M 4/623; H01M 10/4235; H01M 2004/027; H01M 4/587; H01M 4/625; H01M 10/0568; H01M 10/0569

(57) ABSTRACT

A battery that cycles lithium ions includes a negative electrode and an ionically conductive electrolyte. The negative electrode includes electroactive negative electrode particles embedded in a polytetrafluoroethylene matrix. The electrolyte includes an organic solvent, an inorganic lithium salt, and a functional additive consisting of a chemical compound including a bis(trifluoromethanesulfonimide group and a substituted phenyl group.

### CROSS-REFERENCE TO RELATED APPLICATIONS

This application claims the benefit of Chinese Patent Application No. 202310771523.5, filed on Jun. 27, 2023. The entire disclosure of the application referenced above is incorporated herein by reference.

### INTRODUCTION

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against the present disclosure.

The present disclosure relates to electrolyte additives for batteries that cycle lithium ions and, more particularly, to electrolyte additives formulated to form solid electrolyte interfaces on negative electrodes during initial charge of the batteries.

Lithium batteries are used in a wide variety of electronic devices and are a promising candidate to fulfill the requirements of electric vehicles, including hybrid electric vehicles, owing to their high energy and power densities. Secondary lithium batteries generally include a negative electrode, a positive electrode, and an electrolyte that provides a medium for the conduction of lithium ions between the negative and positive electrodes during discharge and charge of the battery. During manufacture, the electrodes are oftentimes deposited in the form of thin layers on electrically conductive metal current collectors. The negative and positive electrode layers may have a composite structure including electrochemically active particles embedded in a polymer binder. One example of a polymer binder that may be beneficially used to manufacture of composite electrodes via a dry process is polytetrafluoroethylene.

### SUMMARY

A battery that cycles lithium ions is disclosed. The battery comprises a positive electrode, a negative electrode spaced apart from the positive electrode, and an electrolyte that provides an ionically conductive pathway for the transport of lithium ions between the positive electrode and the negative electrode. The negative electrode comprises a particulate component embedded in a polymeric matrix component. The particulate component comprises electroactive negative electrode particles and the polymeric matrix component comprises polytetrafluoroethylene. The electrolyte comprises an organic solvent, an inorganic lithium salt, and a functional additive consisting of a chemical compound that comprises a bis(trifluoromethanesulfonimide group and a substituted phenyl group.

The functional additive may consist of a chemical compound having the following formula (1):

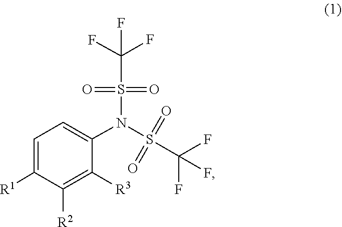

where R¹, R², and R³ are each individually hydrogen, an alkyl group, an alkene group, or a halogen.

During initial charge of the battery, the functional additive may decompose and form a solid electrolyte interface on surfaces of the electroactive negative electrode particles that isolates the electroactive negative electrode particles from physical contact with the polymeric matrix component. And, during subsequent cycling of the battery, the solid electrolyte interface may prevent chemical reactions between the electroactive negative electrode particles and the polytetrafluoroethylene in the polymeric matrix component.

During initial charge of the battery, the functional additive may decompose and form the solid electrolyte interface on surfaces of the electroactive negative electrode particles when the negative electrode is at a potential of greater than or equal to about 1.5 Volts and less than or equal to about 2.2 Volts vs. Li/Li⁺.

The electroactive negative electrode particles may have a solid electrolyte interface formed on surfaces thereof. The solid electrolyte interface may comprise one or more chemical compounds comprising a bis(trifluoromethanesulfonimide group, trifluoromethyl group, sulfur oxide group, or a combination thereof.

The functional additive may constitute, by weight, greater than or equal to about 0.1% to less than or equal to about 5% of the electrolyte.

The inorganic lithium salt may comprise lithium hexafluorophosphate.

The organic solvent may comprise a mixture of ethylene carbonate and ethyl methyl carbonate.

The polymeric matrix component may comprise, by weight, greater than or equal to about 90% polytetrafluoroethylene. The polymeric matrix component may constitute, by weight, greater than or equal to about 1% to less than or equal to about 5% of the negative electrode.

The electroactive negative electrode particles may comprise graphite, silicon, or a combination thereof.

The particulate component may further comprise an electrically conductive carbon-based material.

The polymeric matrix component may be substantially free of polyvinylidene fluoride.

A battery that cycles lithium ions is disclosed. The battery comprises a positive electrode, a negative electrode spaced apart from the positive electrode, and an electrolyte that provides an ionically conductive pathway for the transport of lithium ions between the positive electrode and the negative electrode. The negative electrode comprises electroactive negative electrode particles embedded in a polymeric matrix component. The electroactive negative electrode particles comprise graphite particles, silicon particles, or a combination thereof. The polymeric matrix component comprises polytetrafluoroethylene. The electrolyte comprises an organic solvent, an inorganic lithium salt, and a functional additive consisting of a chemical compound having the following formula (1):

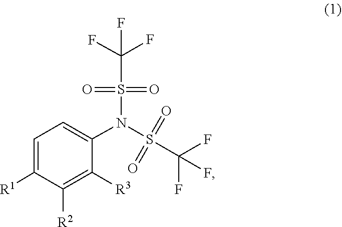

where R¹, R², and R³ are each individually hydrogen, an alkyl group, an alkene group, or a halogen.

During initial charge of the battery, the functional additive may decompose and form a solid electrolyte interface on surfaces of the electroactive negative electrode particles that isolates the electroactive negative electrode particles from physical contact with the polymeric matrix component.

During initial charge of the battery, the functional additive may decompose and form the solid electrolyte interface on surfaces of the electroactive negative electrode particles when the negative electrode is at a potential of greater than or equal to about 1.5 Volts and less than or equal to about 2.2 Volts vs. Li/Li⁺.

The electroactive negative electrode particles may have a solid electrolyte interface formed on surfaces thereof. The solid electrolyte interface may comprise one or more chemical compounds comprising a bis(trifluoromethanesulfonimide group, trifluoromethyl group, sulfur oxide group, or a combination thereof.

The functional additive may constitute, by weight, greater than or equal to about 0.1% to less than or equal to about 5% of the electrolyte.

The inorganic lithium salt may comprise lithium hexafluorophosphate.

The organic solvent may comprise a mixture of ethylene carbonate and ethyl methyl carbonate.

The polymeric matrix component may comprise, by weight, greater than or equal to about 90% polytetrafluoroethylene. The polymeric matrix component may constitute, by weight, greater than or equal to about 1% to less than or equal to about 5% of the negative electrode.

A method of manufacturing a battery is disclosed. The battery comprises a negative electrode and an electrolyte infiltrating the negative electrode. The negative electrode comprises graphite particles embedded in a polytetrafluoroethylene binder. The electrolyte comprises a chemical compound including a bis(trifluoromethanesulfonimide group and a substituted phenyl group. A constant current is applied to the battery at a charge rate of less than or equal to about C/20 until the battery reaches a predetermined maximum potential. Then, a constant voltage is applied to the battery at the predetermined maximum potential until a measured current reaches a charge rate of less than or equal to about C/50.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 is a schematic perspective view of an automotive vehicle powered by battery pack that includes multiple battery modules.

FIG. 2 is a schematic cross-sectional view of a portion of one of the battery modules of FIG. 1, the battery module including multiple battery cells arranged in a stack.

FIG. 3 is a schematic cross-sectional view of a battery that cycles lithium ions, the battery comprising a negative electrode including electroactive negative electrode particles and a polytetrafluoroethylene binder, and an electrolyte including a functional additive that forms a solid electrolyte interphase on the electroactive negative electrode particles during initial charge of the battery.

FIG. 4 is a schematic cross-sectional view of one of the electroactive negative electrode particles of FIG. 3 including a solid electrolyte interphase formed on an exterior surface thereof.

FIGS. 5 and 6 are cyclic voltammograms of measured current (mA) versus applied potential (V vs. Li/Li⁺) for the first cycle of a half coin cell including a baseline electrolyte (dashed line) and a half coin cell including an electrolyte comprising PhTFSI as a functional additive (solid line).

FIG. 7 is a plot of potential (V vs. Li/Li⁺) versus capacity (mAh) depicting charge and discharge profiles for the first cycle of a half coin cell including a baseline electrolyte (dashed line) and a half coin cell including an electrolyte comprising PhTFSI as a functional additive (solid line).

FIG. 8 is a plot of potential (V vs. Li/Li⁺) versus capacity (mAh) depicting charge and discharge profiles for the first cycle of a full cell including a baseline electrolyte (dashed line) and a full cell including an electrolyte comprising PhTFSI as a functional additive (solid line).

In the drawings, reference numbers may be reused to identify similar and/or identical elements.

### DETAILED DESCRIPTION

The presently disclosed electrolytes can be used in batteries that cycle lithium ions to help improve the coulombic efficiency thereof, for example, by preventing undesirable side reactions between the electroactive negative electrode particles and the polytetrafluoroethylene binder in the negative electrode of the battery. Specifically, the presently disclosed electrolytes include a functional additive that is formulated to form a solid electrolyte interface on surfaces of the electroactive negative electrode particles during initial charge of the battery that physically isolates the electroactive negative electrode particles from the polytetrafluoroethylene binder and thereby prevents physical and chemical interactions between the electroactive negative electrode particles and the polytetrafluoroethylene binder in the negative electrode.

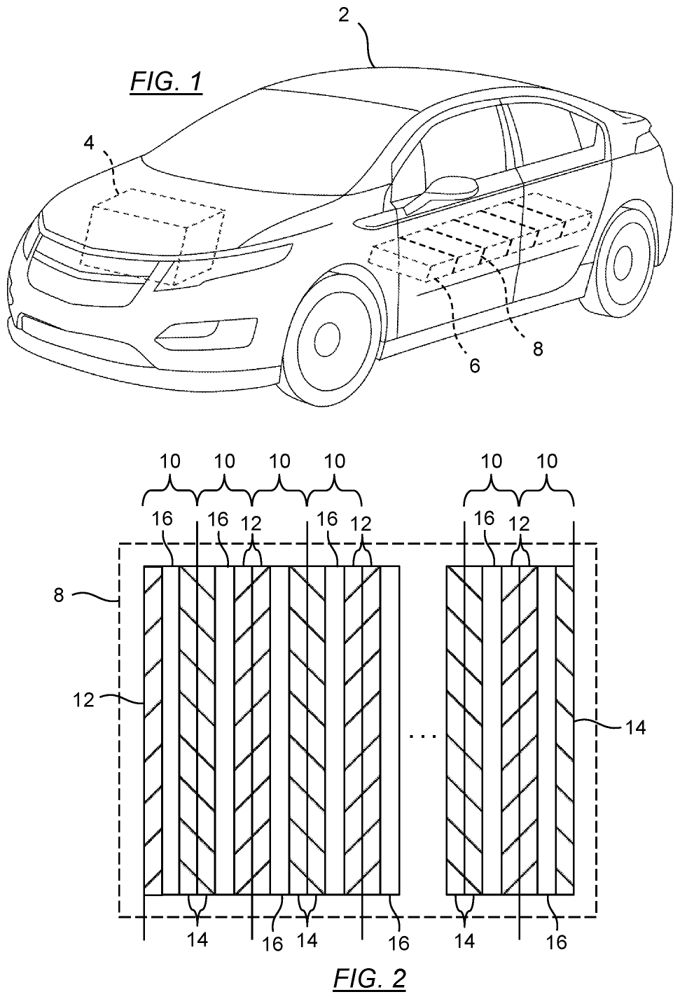

FIG. 1 depicts an automotive vehicle 2 powered by an electric motor 4 that draws electricity from a battery pack 6 including one or more battery modules 8. The battery modules 8 may be electrically coupled together in a series and/or parallel arrangement to meet desired capacity and power requirements of the electric motor 4. The vehicle 2 may be an all-electric vehicle and may be powered exclusively by the electric motor 4, or the vehicle 2 may be a hybrid electric vehicle and may be powered by the electric motor 4 and by an internal combustion engine (not shown).

As shown in FIG. 2, each battery module 8 includes one or more electrochemical cells or batteries 10 that cycle lithium ions. In practice, the batteries 10 in the battery module 8 are oftentimes assembled as a stack of layers, including negative electrode layers 12, negative electrode current collectors 13, positive electrode layers 14, positive electrode current collectors 15, and separator layers 16. Each battery 10 is defined by a negative electrode layer 12 and a positive electrode layer 14, which are spaced apart from each other by a separator layer 16. The negative electrode layers 12 are disposed on and in electrical communication with the negative electrode current collectors 13 and the positive electrode layers 14 are disposed on an in electrical communication with the positive electrode current collectors 15. As shown in FIG. 2, for efficiency, the layers may be stacked such that some of the negative and positive electrode current collectors 13, 15 are double sided and include negative or positive electrode layers 12, 14 on both sides thereof. In this arrangement, adjacent negative electrode layers 12 and positive electrode layers 14 share a single negative or positive current collector 13, 15.

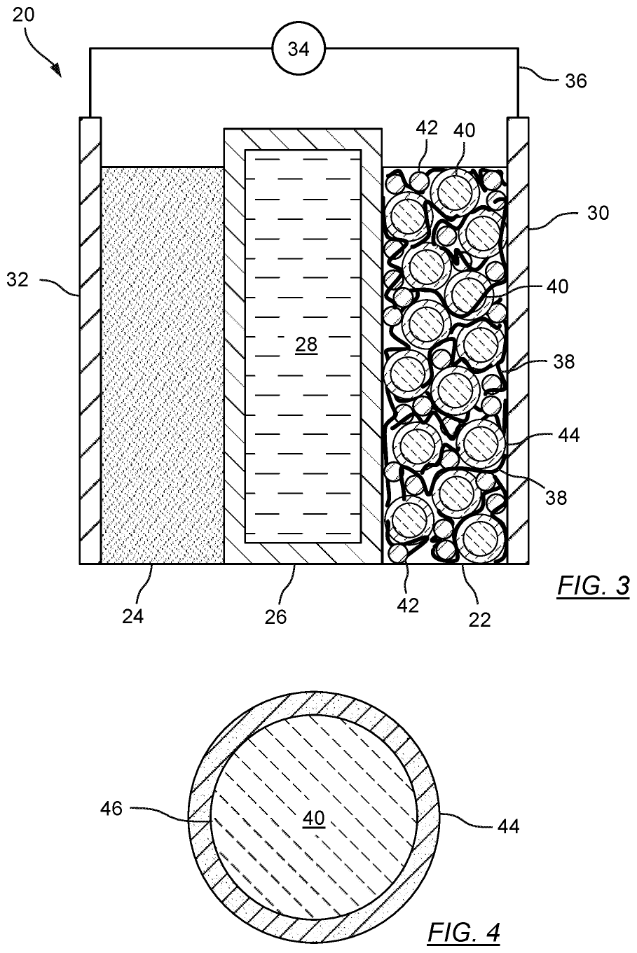

FIG. 3 depicts an electrochemical cell or battery 20 that cycles lithium ions. The battery 20 can generate an electric current during discharge, which may be used to supply power to a load device (e.g., an electric motor 4), and can be charged by being connected to a power source. Like the batteries 10 depicted in FIGS. 1 and 2, in aspects, the battery 20 may be used to supply power to an electric motor 4 of an automotive vehicle 2. Additionally or alternatively, the battery 20 may be used in other transportation applications (e.g., motorcycles, boats, tractors, buses, motorcycles, mobile homes, campers, tanks, and aircraft), and may be used to provide electricity to stationary and/or portable electronic equipment, components, and devices used in a wide variety of other industries and applications, including industrial, residential, and commercial buildings, consumer products, industrial equipment and machinery, agricultural or farm equipment, and heavy machinery, by way of nonlimiting example.

The battery 20 comprises a negative electrode 22, a positive electrode 24, a separator 26, and an electrolyte 28 that infiltrates the negative electrode 22, the positive electrode 24, and the separator 26. The negative electrode 22 is disposed on a negative electrode current collector 30 and the positive electrode 24 is disposed on a positive electrode current collector 32. In practice, the negative electrode current collector 30 and the positive electrode current collector 32 are electrically coupled to a power source or load 34 (e.g., the electric motor 12) via an external circuit 36. The negative and positive electrodes 22, 24 are formulated such that, when the battery 20 is at least partially charged, an electrochemical potential difference is established between the negative and positive electrodes 22, 24. During discharge of the battery 20, the electrochemical potential established between the negative and positive electrodes 22, 24 drives spontaneous redox reactions within the battery 20 and the release of lithium ions and electrons at the negative electrode 22. The released lithium ions travel from the negative electrode 22 to the positive electrode 24 through the separator 26 and the electrolyte 28, and the electrons travel from the negative electrode 22 to the positive electrode 24 via the external circuit 36, which generates an electric current. After the negative electrode 22 has been partially or fully depleted of lithium, the battery 20 may be charged by connecting the negative and positive electrodes 22, 24 to the power source 34, which drives nonspontaneous redox reactions within the battery 20 and the release of the lithium ions and the electrons from the positive electrode 24. The repeated discharge and charge of the battery 20 may be referred to herein as “cycling,” with a full charge event followed by a full discharge event being considered a full cycle.

The negative electrode 22 is a porous composite material and includes a polymeric matrix component 38 and a particulate component embedded in the polymeric matrix component 38. The particulate component includes electrochemically active (electroactive) particles 40 and optionally electrically conductive particles 42 distributed throughout the polymeric matrix component 38. The polymeric matrix component 38 and the particulate component of the negative electrode 22 may define a plurality of open pores and may provide the negative electrode 22 with a porosity of greater than or equal to about 20% to less than or equal to about 50%. The negative electrode 22 may have a density of greater than or equal to about 1.3 grams per cubic centimeter (g/cm³) to less than or equal to about 2 g/cm³.

The polymeric matrix component 38 is formulated to act as a binder and provide the negative electrode 22 with structural integrity, for example, by creating cohesion between the electroactive particles 40 and the optional electrically conductive particles 42. The polymeric matrix component 38 comprises polytetrafluoroethylene (PTFE). The polymeric matrix component 38 may comprise, by weight, greater than about 80%, greater than about 90%, greater than about 95%, or greater than about 99% polytetrafluoroethylene. In aspects, the polymeric matrix component 38 may consist essentially of or consist entirely of polytetrafluoroethylene. The polymeric matrix component 38 may account for, by weight, greater than or equal to about 0.5%, or optionally about 1%, to less than or equal to about 10%, optionally about 5%, or optionally about 3% of the negative electrode 22. In aspects, the polymeric matrix component 38 may account for, by weight, about 1.5% of the negative electrode 22.

The electroactive particles 40 can store and release lithium ions during charge and discharge, respectively, of the battery 20. The electroactive particles 40 comprise one or more electrochemically active (electroactive) materials that can undergo a reversible redox reaction with lithium during charge and discharge of the battery 20. For example, the electroactive particles 40 may comprise a lithium intercalation host material, a material that can reversibly alloy with lithium (a lithium alloying material), and/or a conversion material that can reversibly react with lithium by undergoing a phase change or a change in crystalline structure accompanied by a change in oxidation state during charging and discharge of the battery 20. Examples of lithium intercalation host materials, lithium alloying materials, and conversion materials include: carbon-based materials (e.g., graphite, graphene, and/or carbon nanotubes), silicon-based materials (e.g., silicon (Si), Si alloys, and/or silicon oxide (SiOx, where 0<x<2), lithiated silicon oxide (LiySiOx, where 0<x<2, 0<y<1)), tin-based materials (e.g., tin (Sn) and/or Sn alloys), metal oxides (e.g., V₂O₅ and/or Co₃O₄), and/or metal sulfides (e.g., FeS). In aspects, the electroactive particles 40 may comprise a combination of carbon-based materials and silicon-based materials. For example, the electroactive particles 40 may comprise a combination of graphite and silicon. In aspects, the electroactive particles 40 may comprise, by weight, greater than or equal to about 50%, greater than or equal to about 75%, or greater than or equal to about 90% carbon-based materials (e.g., graphite). In aspects, the electroactive particles 40 may comprise, by weight, greater than or equal to about 1% to less than or equal to about 50%, or optionally about 15% silicon. The electroactive particles 40 may constitute, by weight, greater than or equal to about 90%, or optionally about 95%, to less than or equal to about 99%, or optionally about 98% of the negative electrode 22. The electroactive particles 40 may provide the negative electrode 22 with an areal capacity of greater than or equal to about 3.5 milliampere-hours per square centimeter (mAh/cm²) to less than or equal to about 7.5 mAh/cm².

As shown in FIGS. 3 and 4, after initial charge and/or after the formation cycle of the battery 20, the electroactive particles 40 include a solid electrolyte interface 44 formed in situ on surfaces 46 thereof. The solid electrolyte interface 44 is ionically conductive and electrically insulating and allows lithium ions to pass therethrough during charge and discharge of the battery 20. In aspects, the solid electrolyte interface 44 may be formed over an around substantially the entire exterior surface of each of the electroactive particles 40. In such case, each of the electroactive particles 40 may be entirely encapsulated by the solid electrolyte interface 44.

The optional electrically conductive particles 42 are formulated to provide the negative electrode 22 with high electrical conductivity, for example, by forming a robust electrically conductive network throughout the negative electrode 22. The electrically conductive particles 42 are electrochemically inactive and do not reversibly react with lithium during charge and discharge of the battery 20. The electrically conductive particles 42 may comprise particles of a carbon-based material, metal particles, and/or an electrically conductive polymer. Examples of electrically conductive carbon-based materials include carbon black (e.g., acetylene black), graphite, graphene (e.g., graphene nanoplatelets), carbon nanotubes (e.g., single-walled and/or multiwalled carbon nanotubes), carbon fibers (e.g., carbon nanofibers), and/or Ketjenblack®. Examples of electrically conductive metal particles include powdered copper, nickel, aluminum, silver, and/or alloys thereof. Examples of electrically conductive polymers include polyaniline, polythiophene, polyacetylene, and/or polypyrrole. The optional electrically conductive particles 42 may constitute for, by weight, greater than 0%, or optionally about 0.5%, to less than or equal to about 5%, or optionally about 1%, of the negative electrode 22. For example, the electrically conductive particles 42 may account for, by weight, about 0.8% of the negative electrode 22.

The positive electrode 24 can store and release lithium ions during discharge and charge of the battery 20. The positive electrode 24 comprises an electroactive material that can undergo a reversible redox reaction with lithium at a higher electrochemical potential than the electroactive particles 40 of the negative electrode 22 such that an electrochemical potential difference exists between the negative electrode 22 and the positive electrode 24. For example, the positive electrode 24 may comprise an electroactive material that can sufficiently undergo lithium intercalation and deintercalation, alloying and dealloying, or plating and stripping. In one form, the electroactive material of the positive electrode 24 may comprise an intercalation host material that can undergo the reversible insertion or intercalation of lithium ions. In such case, the electroactive material of the positive electrode 24 may comprise a lithium transition metal oxide. For example, the electroactive material of the positive electrode 24 may comprise a layered oxide represented by the formula LiMeO₂, an olivine-type oxide represented by the formula LiMePO₄, a spinel-type oxide represented by the formula LiMe₂O₄, a tavorite represented by one or both of the following formulas LiMeSO₄F or LiMePO₄F, or a combination thereof, where Me is a transition metal (e.g., Co, Ni, Mn, Fe, Al, V, or a combination thereof). In some embodiments, the electroactive material of the positive electrode 24 may comprise a lithium transition metal oxide selected from the group consisting of lithium cobalt oxide (LCO), lithium manganese oxide (LMO), lithium nickel manganese cobalt oxide (NMC), lithium nickel cobalt aluminum oxide (NCA), lithium nickel cobalt manganese aluminum oxide (NCMA), and lithium manganese iron phosphate (LMFP). In another form, electroactive material of the positive electrode 24 may comprise a conversion material including a component that can undergo a reversible electrochemical reaction with lithium, in which the component undergoes a phase change or a change in crystalline structure accompanied by a change in oxidation state. In such case, the electroactive material of the positive electrode 24 may comprise sulfur, selenium, tellurium, iodine, a halide (e.g., a fluoride or chloride), sulfide, selenide, telluride, iodide, phosphide, nitride, oxide, oxysulfide, oxyfluoride, sulfur-fluoride, sulfur-oxyfluoride, or a lithium and/or metal compound thereof. Examples of suitable metals for inclusion in the conversion material of the electroactive material of the positive electrode 24 include iron, manganese, nickel, copper, and cobalt.

The electroactive material of the positive electrode 24 may be intermingled with an electrically conductive agent and a polymer binder. The electrically conductive agent may comprise any of the materials discussed above with respect to the electrically conductive particles 42. The polymer binder may comprise an organic polymer. Examples of organic polymers include polytetrafluoroethylene (PTFE), polyvinylidene fluoride (PVDF), ethylene propylene diene monomer (EPDM) rubber, styrene butadiene rubber (SBR), carboxymethyl cellulose (CMC), and/or polyacrylic acid (PAA).

The separator 26 physically and electrically isolates the negative electrode 22 and the positive electrode 24 from each other while permitting lithium ions to pass therethrough. The separator 26 has an open microporous structure and may comprise an organic and/or inorganic material. For example, the separator 26 may comprise a polymer or a combination of polymers. For example, the separator 26 may comprise one or more polyolefins, e.g., polyethylene (PE), polypropylene (PP), polyamide (PA), poly(tetrafluoroethylene) (PTFE), polyvinylidene fluoride (PVdF), and/or poly(vinyl chloride) (PVC). In one form, the separator 26 may comprise a laminate of polymers, e.g., a laminate of PE and PP. In aspects, the separator 26 may comprise a ceramic coating (not shown) disposed on one or both sides thereof. In such case, the ceramic coating may comprise particles of alumina (Al₂O₃) and/or silica (SiO₂).

The negative and positive electrode current collectors 30, 32 are electrically conductive and provide an electrical connection between the external circuit 36 and their respective negative and positive electrodes 22, 24. In aspects, the negative and positive electrode current collectors 30, 32 may be in the form of nonporous metal foils, perforated metal foils, porous metal meshes, or a combination thereof. The negative electrode current collector 30 may be made of copper, nickel, or alloys thereof, stainless steel, or other appropriate electrically conductive material. The positive electrode current collector 32 may be made of aluminum (AI) or another appropriate electrically conductive material.

The electrolyte 28 is ionically conductive and provides a medium for the conduction of lithium ions between the negative electrode 22 and the positive electrode 24. In assembly, the battery 20 is infiltrated with the electrolyte 28 such that the electrolyte 28 is in direct physical contact with the negative electrode 22, the positive electrode 24, and the separator 26. For example, the electrolyte 28 may infiltrate open pores of the negative electrode 22, the positive electrode 24, and the separator 26. The electrolyte 28 is formulated to provide the battery 20 with enhanced cycling stability by promoting the in situ formation of the solid electrolyte interface 44 on the surfaces 46 of the electroactive particles 40 of the negative electrode 22 during the initial charge of the battery 20. The electrolyte 28 may comprise an organic solvent, optionally one or more co-solvents, a lithium salt, and a functional additive.

The organic solvent may comprise a nonaqueous aprotic organic solvent. For example, the organic solvent may comprise an alkyl carbonate, aliphatic carboxylic ester, γ-lactone, or a combination thereof. In aspects, the organic solvent may comprise a mixture of alkyl carbonates, for example, a mixture of a cyclic carbonate and a linear carbonate. Examples of cyclic carbonates include ethylene carbonate (EC), propylene carbonate (PC), butylene carbonate (BC), fluoroethylene carbonate (FEC), and combinations thereof. Examples of linear carbonates include dimethyl carbonate (DMC), diethyl carbonate (DEC), ethyl methyl carbonate (EMC), and combinations thereof. Examples of aliphatic carboxylic esters include methyl formate, methyl acetate, methyl propionate, and combinations thereof. Examples of γ-lactones include γ-butyrolactone, γ-valerolactone, and combinations thereof.

The organic solvent may constitute, by weight, greater than or equal to about 70% to less than or equal to about 90% of the electrolyte 28. The volumetric ratio of the cyclic carbonate to the linear carbonate in the organic solvent may be greater than or equal to about 2 to 8 (cyclic carbonate:linear carbonate=2:8) and less than or equal to about 4 to 6 (4:6). In aspects, the volumetric ratio of the cyclic carbonate to the linear carbonate in the organic solvent may be about 3:7. In aspects, the organic solvent may comprise a mixture of ethylene carbonate (a cyclic carbonate) and ethyl methyl carbonate (a linear carbonate). In such case, the ethylene carbonate may constitute, by weight, greater than or equal to about 20% or optionally about 25% to less than or equal to about 40% or optionally about 35% of the electrolyte 28. In aspects, the ethylene carbonate may constitute, by weight, about 30% of the electrolyte 28. The ethyl methyl carbonate may constitute, by weight, greater than or equal to about 45% or optionally about 50% to less than or equal to about 70%, optionally about 65%, or optionally about 60% of the electrolyte 28. In aspects, the ethyl methyl carbonate may constitute, by weight, about 55% of the electrolyte 28.

The one or more co-solvents may comprise vinylene carbonate (VC), 1,3-propane sultone (PS), 1,3,2-dioxathiolane 2,2-dioxide (DTD), and combinations thereof.

The lithium salt is soluble in the organic solvent and provides a passage for lithium ions through the electrolyte 28. The lithium salt may comprise an inorganic lithium salt, an organic lithium salt, or a combination thereof. Examples of inorganic lithium salts include lithium hexafluorophosphate (LiPF₆), lithium perchlorate (LiClO₄), lithium tetrachloroaluminate (LiAlCl₄), lithium iodide (Lil), lithium bromide (LiBr), lithium thiocyanate (LiSCN), lithium tetrafluoroborate (LiBF₄), lithium tetraphenylborate (LiB(C₆H₅)₄), lithium bis(oxalato)borate (LiB(C₂O₄)₂) (LiBOB), lithium difluorooxalatoborate (LiBF₂(C₂O₄)), lithium hexafluoroarsenate (LiAsF₆), lithium trifluoromethanesulfonate (LiCF₃SO₃), lithium bis(trifluoromethane)sulfonylimide (LiN(CF₃SO₂)₂), lithium bis(fluorosulfonyl)imide (LiN(FSO₂)₂) (LiFSI), lithium bis(trifluoromethanesulfonyl)imide (LiTFSI), and combinations thereof. The lithium salt may be dissolved in the organic solvent at a concentration of greater than or equal to about 0.5 Molar, or optionally about 0.6 Molar and less than or equal to about 2.0 Molar, or optionally about 1.5 Molar. In aspects, the lithium salt may be dissolved in the organic solvent at a concentration of about 1 Molar. The lithium salt may constitute, by weight, greater than or equal to about 5%, optionally about 8%, or optionally about 10% to less than or equal to about 20%, optionally about 15%, or optionally about 12% of the electrolyte 28. In aspects, the lithium salt may constitute, by weight, about 11% of the electrolyte 28. In aspects, the lithium salt may comprise LiPF₆.

The functional additive is formulated to allow for the in situ formation of the solid electrolyte interface 44 on the surfaces 46 of the electroactive particles 40 of the negative electrode 22 during the initial charge and/or the formation cycle of the battery 20.

The functional additive comprises a chemical compound comprising a bis(trifluoromethanesulfonimide (TFSI) group and a substituted phenyl group, e.g., a monosubstituted phenyl, a disubstituted phenyl, a trisubstituted phenyl, or a combination thereof. The functional additive comprises a chemical compound having the following formula (1):

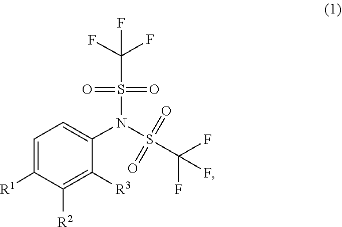

where R¹, R², and R³ are each individually hydrogen (H), an alkyl group (CₙH₂ₙ₊₁) or an alkene group (CₙH₂ₙ), where n is an integer greater than or equal to 1 and less than or equal to 20, or a halogen (e.g., Cl, F, Br, and/or I). Alternatively, the chemical compound of formula (1) may be represented by the following linear formula: R¹R²R³C₆H₂N(SO₂CF₃)₂.

In aspects, in the chemical compound of formula (1), R¹, R², and R³ each may be hydrogen and the functional additive may comprise N-Phenyl-bis(trifluoromethanesulfonimide), CAS No. 37595-74-7.

The functional additive may constitute, by weight, greater than or equal to about 0.1%, optionally about 0.3%, or optionally about 0.5% to less than or equal to about 5%, optionally about 3%, or optionally about 1.5% of the electrolyte 28.

The solid electrolyte interface 44 is formed in situ on the surfaces 46 of the electroactive particles 40 of the negative electrode 22 during the initial charge and/or during the formation cycle of the battery 20 due to chemical reactions between the functional additive in the electrolyte 28 and the electroactive particles 40 of the negative electrode 22. After initial assembly of the battery 20, the potential at the negative electrode 22 may be greater than or equal to about 2 Volts vs. Li/Li⁺. During the initial charge of the battery 20, lithium is intercalated or otherwise inserted into the electroactive particles 40 of the negative electrode 22, which gradually decreases the potential at the negative electrode 22. As the potential of the negative electrode 22 decreases below the reduction potential of the functional additive, the functional additive in the electrolyte 28 decomposes via electrochemical reduction reactions and the decomposition products thereof deposit on the surfaces 46 of the electroactive particles 40 to form the solid electrolyte interface 44. The reduction potential (vs. Li/Li⁺) of the functional additive may be greater than or equal to about 1.5 Volts, or optionally about 1.8 Volts, and less than or equal to about 2.2 Volts, or optionally about 2 Volts. In aspects, the reduction potential of the functional additive may be about 1.9 Volts vs. Li/Li⁺. As such, during initial charge of the battery 20, when the potential of the negative electrode 22 is decreased below about 2.2 Volts vs. Li/Li⁺, for example, to less than or equal to about 2 Volts vs. Li/Li⁺, the functional additive decomposes and forms the solid electrolyte interface 44 on the surfaces 46 of the electroactive particles 40.

The solid electrolyte interface 44 formed in situ on the electroactive particles 40 comprises products of the electrochemical reduction of the chemical compound of formula (1). For example, the solid electrolyte interface 44 may comprise one or more chemical compounds that have a bis(trifluoromethanesulfonimide group (—N(SO₂CF₃)₂), trifluoromethyl group (—CF₃), sulfur oxide group (—SOx), or a combination thereof. In aspects, the solid electrolyte interface 44 may be substantially free of lithium fluoride (LiF), lithium-, phosphorus-, and fluorine-containing compounds (e.g., LiₓPF_{y}), lithium-, phosphorus-, fluorine-, and oxygen-containing compounds (e.g., LiₓPF_{y}O_{z}).

There are several notable benefits of using PTFE as the polymeric matrix component 38 of the negative electrode 22. For example, using PTFE as the polymeric matrix component 38 of the negative electrode 22 may allow for the formation of a relatively thick negative electrode 22 with robust cohesive properties. In addition, when PTFE is used as the polymeric matrix component 38 of the negative electrode 22, the negative electrode 22 may be formed via a dry process (e.g., a hot pressing process) and thus may eliminate the need to use potentially toxic N-Methylpyrrolidone (NMP) as a solvent in the manufacturing process, thereby reducing manufacturing costs. Further, the high thermal and chemical resistance of PTFE may help extend the life of the battery 20. What's more, the PTFE may be fibrillated and, in such case, the PTFE fibers may make point or line contact with the surfaces 46 of the electroactive particles 40 while permitting flow of lithium ions between the electroactive particles 40 and the electrolyte 28, which may improve the rate capability of the battery 20. In addition, when fibrillated, the PTFE fibers may help form a strong cohesive network that bonds together both the electroactive particles 40 and the electrically conductive particles 42 in the negative electrode 22. The robust and flexible architecture and mechanical strength provided by using PTFE as the as the polymeric matrix component 38 of the negative electrode 22 can be maintained during the life of the battery 20 to improve the cycling stability. However, parasitic side reactions may occur between the PTFE and the lithium stored in the electroactive particles 40 during charge of the battery 20 due to the low potential reached at the negative electrode 22 during charge. And such side reactions may consume active lithium and thereby reduce the overall capacity of the battery 20.

The inventors of the present disclosure have discovered that formation of the solid electrolyte interface 44 on the surfaces 46 of the electroactive particles 40 helps prevent or inhibit undesirable chemical reactions from occurring between the electroactive particles 40 and the polytetrafluoroethylene in the polymeric matrix component 38 during cycling of the battery 20. In particular, formation of the solid electrolyte interface 44 on the surfaces 46 of the electroactive particles 40 creates a physical barrier that shields the electroactive particles 40 from physical contact with the polytetrafluoroethylene in the polymeric matrix component 38.

Notably, the inventors of the present disclosure have discovered that the reduction potential of the functional additive is higher than that of the polytetrafluoroethylene in the polymeric matrix component 38 of the negative electrode 22. Therefore, the solid electrolyte interface 44 will form on the surfaces 46 of the electroactive particles 40 during initial charge of the battery 20 before the potential of the negative electrode 22 is reduced to level which would be low enough initiate decomposition of the polytetrafluoroethylene in the polymeric matrix component 38. Specifically, the inventors of the present disclosure have discovered that undesirable side reactions between polytetrafluoroethylene in the polymeric matrix component 38 and the electroactive particles 40 in the negative electrode 22 do not occur during charge of the battery 20 until the potential of the negative electrode 22 is decreased to less than or equal to about 0.5 Volts vs. Li/Li⁺, which is significantly lower than the potential at which the functional additive in the electrolyte 28 decomposes to form the solid electrolyte interface 44 (e.g., about 1.9 Volts vs. Li/Li⁺).

After initial assembly of the battery 20, the negative electrode 22 may be substantially free of lithium and may be subjected to charge and discharge protocols to ensure formation of the solid electrolyte interface 44 on the surfaces 46 of the electroactive particles 40. For example, a constant current and constant voltage (CCCV) protocol may be used to charge the battery 20. The CCCV charge protocol may include applying a constant current to the battery 20 at a charge rate of less than or equal to about C/20 until the battery 20 reaches a predetermined maximum potential, Vmax (e.g., of about 4.2V). Then a constant voltage at the predetermined maximum potential, Vmax, may be applied to the battery 20 until the current reaches a charge rate of less than or equal to about C/50. The battery 20 may be subsequently discharged to a predetermined minimum potential, Vmin (e.g., about 2.7V) at a discharge rate of about C/5.

### EXPERIMENTAL

### Half Coin Cell Preparation

Half coin cells including graphite electrodes were assembled and evaluated using cyclic voltammetry (CV) testing and galvanostatic charge and discharge protocols. The graphite electrodes were prepared via a hot-pressing process, had an areal capacity of about 5.5 mAh/cm², were deposited on 8-micron copper (Cu) foil current collectors, dried in a vacuum at 140 degrees Celsius (° C.), and cut into 14 millimeter (mm) discs. The graphite electrodes comprised a mixture of graphite (G), carbon nanotubes (CNT), and PTFE at a G:CNT:PTFE mass ratio of 97.7:0.8:1.5. Electrolytes were prepared comprising a mixture of ethylene carbonate (EC) and ethyl methyl carbonate (EMC) at an EC:EMC mass ratio of 3:7, LiPF₆ at a concentration of 1 Molar, and 2% vinylene carbonate (VC), based on the total weight of the electrolyte (baseline electrolyte). Electrolytes in accordance with embodiments of the present disclosure were prepared by adding to the baseline electrolyte an additive consisting of N-Phenyl-bis(trifluoromethanesulfonimide) (PhTFSI) at a concentration of 3%, based upon the total weight of the electrolyte (PhTFSI-electrolyte).

### Cyclic Voltammetry Testing of Half Coin Cells

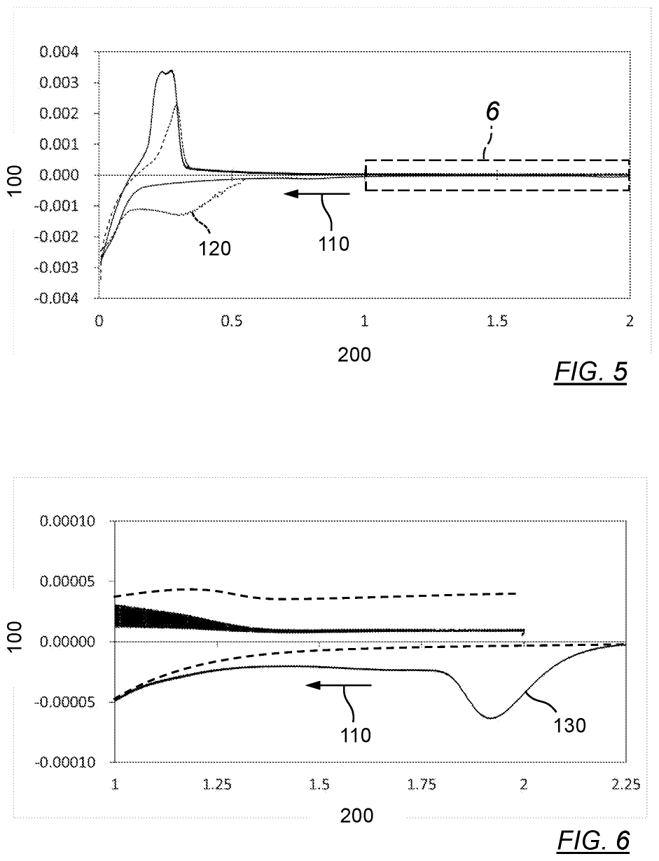

Cyclic voltammetry (CV) testing was performed on the half coin cells including the graphite electrodes using a 0.1 millivolt per second (mV/s) scan rate at a temperature of about 25° C. FIGS. 5 and 6 are cyclic voltammograms of measured current (milliampere, mA) 100 versus applied potential (Volts, V vs. Li/Li⁺) 200 for the first cycle of a half coin cell including the baseline electrolyte (dashed line) and a half coin cell including the PhTFSI-electrolyte (solid line). During the CV testing, the potential was swept negatively from about 2.5 V to 0 V, with the arrow 110 indicating the sweep direction of the forward scan. The curves with measured current values less than 0 mA on the γ-axis depict the reduction reactions occurring at the graphite electrodes during the charging process and the intercalation of lithium ions into the graphite electrodes.

As shown in FIG. 5, the measured current for the half coin cell including the baseline electrolyte (dashed line) exhibits a reduction peak 120 starting at about 0.5 V, signifying the occurrence of a side reaction occurring between PTFE and the lithiated graphite (LiCx) at a potential of about 0.5 V during the charging process. FIG. 6 is an inset of FIG. 5. As shown in FIG. 6, the measured current for the half coin cell including the PhTFSI-electrolyte (solid line) exhibits a reduction peak 130 starting at around 2.2 V and peaking at about 1.9 V. Without intending to be bound by theory, it is believed that the reduction peak 130 indicates the occurrence of a reduction reaction occurring between the PhTFSI additive in the electrolyte and the lithiated graphite at a potential of about 2.2 V during the charging process and the formation of the solid electrolyte interface 44, which inhibits the side reaction from subsequently occurring between the PTFE and the lithiated graphite at a potential of about 0.5 V, as evidenced by the absence of a reduction peak at 0.5 V in FIG. 5 for the half coin cell including the PhTFSI-electrolyte (FIG. 5).

### Galvanostatic Charge and Discharge Testing of Half Coin Cells

Half coin cells including the graphite electrodes were galvanostatically charged and discharged in a voltage range of 10 millivolts (mV) to 2 V using a Maccor cycler at room temperature (e.g., 25° C.). A constant current and constant voltage (CCCV) protocol was used to charge the half coin cells at a constant current using a C/20 charge rate to a potential of about 10 mV, then constant voltage charge at 10 mV until the current reached C/50. The half coin cells were subsequently discharged to 2 V at C/20 rate.

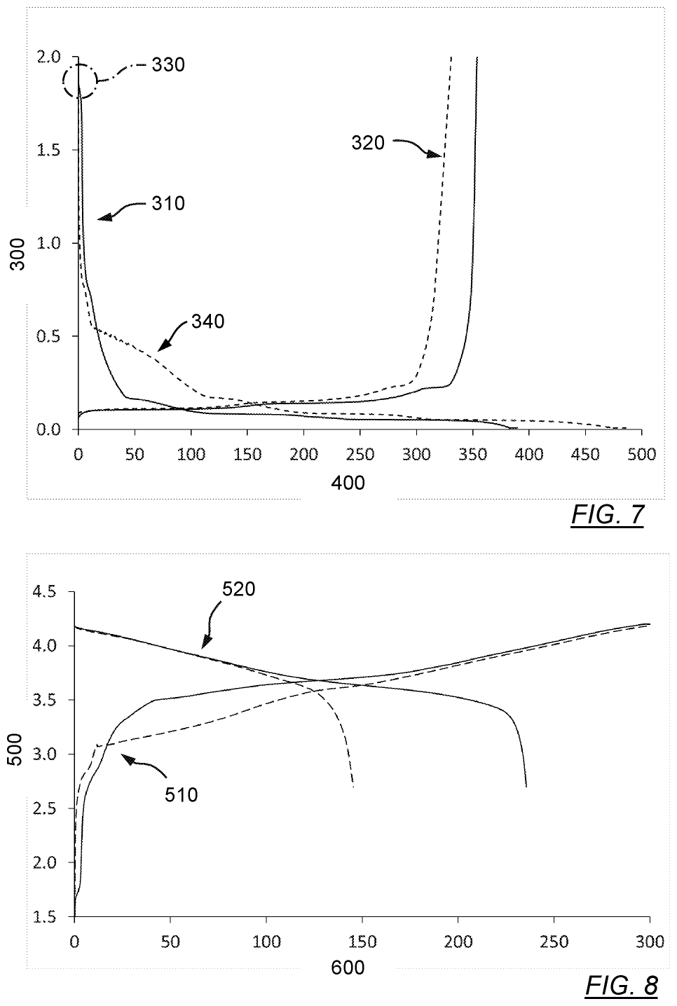

FIG. 7 is a plot of potential (V vs. Li/Li⁺) 300 versus capacity (mAh) 400 depicting charge profiles 310 and discharge profiles 320 for the first cycle of a half coin cell including the baseline electrolyte (dashed line) and a half coin cell including the PhTFSI-electrolyte (solid line). The circle 330 at a potential of about 1.9 V on the charge profile 310 indicates the expected point during the charge process where the PhTFSI additive in the electrolyte decomposes to form the solid electrolyte interface 44. As shown in FIG. 7, the half coin cell including the PhTFSI-electrolyte exhibited a significantly higher coulombic efficiency (CE) (CE of about 91%) than the half coin cell including the baseline electrolyte (CE of about 68%). In addition, the electrochemical performance and efficiency of the charge cycle of the half coin cell including the baseline electrolyte was degraded by the side reactions occurring between PTFE and the lithiated graphite (LiCx) (indicated by arrow 340) starting at a potential of about 0.5 V.

### Full Cell Preparation and Testing

Full pouch cells including graphite negative electrodes were assembled and evaluated using galvanostatic charge and discharge protocols. The composition of the graphite negative electrodes and electrolytes were the same as described above for the half coin cells. The positive electrodes had an areal capacity of about 4.75 mAh/cm² and comprised a mixture of LiNi_{0.75}Mn_{0.25}O₂ (NM75), Super P carbon black (SP), Ketjenblack (KB), and PTFE at a NM75:SP:KB:PTFE mass ratio of 96:1:1:2.

A constant current and constant voltage (CCCV) protocol was used to charge the full cells at a constant current using a C/20 charge rate to a potential of about 4.2V, then constant voltage charge at 4.2V until the current reached C/50. The full cells were subsequently discharged to 2.7 V at C/5 rate. FIG. 8 is a plot of potential (V vs. Li/Li⁺) 500 versus capacity (mAh) 600 depicting charge profiles 510 and discharge profiles 520 for the first cycle of a full cell including the baseline electrolyte (dashed line) and a full cell including the PhTFSI-electrolyte (solid line). As shown in FIG. 8, the full cell including the PhTFSI-electrolyte exhibited a significantly higher coulombic efficiency (CE of about 78%) than the full cell including the baseline electrolyte (CE of about 48%). In addition, the full cell including the PhTFSI-electrolyte had a higher overall capacity than the full cell including the baseline electrolyte.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

The terminology used herein is for the purpose of describing example embodiments only and is not intended to be limiting. As used herein, the singular forms (“a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended terms “comprises,” “comprising,” “including,” and “having,” are to be understood as non-restrictive terms used to describe and claim various embodiments set forth herein, in certain aspects, the terms may alternatively be understood to instead be a more limiting and restrictive term, such as “consisting of” or “consisting essentially of.” Thus, for any given embodiment reciting compositions, materials, components, elements, ingredients, features, integers, operations, and/or process steps, the present disclosure also specifically includes embodiments consisting of, or consisting essentially of, such recited compositions, materials, components, elements, ingredients, features, integers, operations, and/or process steps. In the case of “consisting of,” the alternative embodiment excludes any additional compositions, materials, components, elements, ingredients, features, integers, operations, and/or process steps, while in the case of “consisting essentially of,” any additional compositions, materials, components, elements, ingredients, features, integers, operations, and/or process steps that materially affect the basic and novel characteristics are excluded from such an embodiment, but any compositions, materials, components, elements, ingredients, features, integers, operations, and/or process steps that do not materially affect the basic and novel characteristics can be included in the embodiment.

Any method steps, processes, and operations described herein are not to be construed as necessarily requiring their performance in the order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer, or section. Terms such as “first,” “second,” and other numerical terms when used herein do not imply a sequence or order unless clearly indicated by the context. Thus, a first step, element, component, region, layer, or section discussed below could be termed a second step, element, component, region, layer, or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s), as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges and encompass minor deviations from the given values and embodiments, having about the value mentioned as well as those having exactly the value mentioned. Other than the working examples provided at the end of the detailed description, all numerical values of parameters (e.g., of quantities or conditions) in this specification are to be understood as being modified in all instances by the term “about” whether or not “about” actually appears before the numerical value. Numerical values of parameters in the appended claims are to be understood as being modified by the term “about” only when such term appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%. In addition, disclosure of ranges includes disclosure of all values and further divided ranges within the entire range, including endpoints and sub-ranges given for the ranges.

As used herein, the terms “composition” and “material” are used interchangeably to refer broadly to a substance containing at least the preferred chemical constituents, elements, or compounds, but which may also comprise additional elements, compounds, or substances, including trace amounts of impurities, unless otherwise indicated. An “X-based” composition or material broadly refers to compositions or materials in which “X” is the single largest constituent of the composition or material on a weight percentage (%) basis. This may include compositions or materials having, by weight, greater than 50% X, as well as those having, by weight, less than 50% X, so long as X is the single largest constituent of the composition or material based upon its overall weight. When a composition or material is referred to as being “substantially free” of a substance, the composition or material may comprise, by weight, less than 5%, optionally less than 3%, optionally less than 1%, or optionally less than 0.1% of the substance.

As used herein, the term “metal” may refer to a pure elemental metal or to an alloy of an elemental metal and one or more other metal or nonmetal elements (referred to as “alloying” elements). The alloying elements may be selected to impart certain desirable properties to the alloy that are not exhibited by the base metal element.

What is claimed is:

1. A battery that cycles lithium ions, the battery comprising:
a positive electrode;
a negative electrode spaced apart from the positive electrode, the negative electrode comprising a particulate component embedded in a polymeric matrix component, the particulate component comprising electroactive negative electrode particles and the polymeric matrix component comprising polytetrafluoroethylene; and
an electrolyte that provides an ionically conductive pathway for the transport of lithium ions between the positive electrode and the negative electrode, the electrolyte comprising:
an organic solvent;
an inorganic lithium salt; and
a functional additive consisting of a chemical compound that comprises a bis(trifluoromethanesulfonimide group and a substituted phenyl group.

2. The battery of claim 1, wherein the functional additive consists of a chemical compound having the following formula (1): 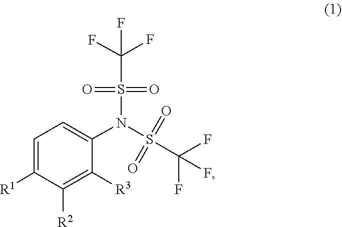
where R¹, R², and R³ are each individually hydrogen, an alkyl group, an alkene group, or a halogen.

3. The battery of claim 1, wherein, during initial charge of the battery, the functional additive decomposes and forms a solid electrolyte interface on surfaces of the electroactive negative electrode particles that isolates the electroactive negative electrode particles from physical contact with the polymeric matrix component, and wherein, during subsequent cycling of the battery, the solid electrolyte interface prevents chemical reactions between the electroactive negative electrode particles and the polytetrafluoroethylene in the polymeric matrix component.

4. The battery of claim 3, wherein, during initial charge of the battery, the functional additive decomposes and forms the solid electrolyte interface on surfaces of the electroactive negative electrode particles when the negative electrode is at a potential of greater than or equal to about 1.5 Volts and less than or equal to about 2.2 Volts vs. Li/Li⁺.

5. The battery of claim 1, wherein the electroactive negative electrode particles have a solid electrolyte interface formed on surfaces thereof, and wherein the solid electrolyte interface comprises one or more chemical compounds comprising a bis(trifluoromethanesulfonimide group, trifluoromethyl group, sulfur oxide group, or a combination thereof.

6. The battery of claim 1, wherein the functional additive constitutes, by weight, greater than or equal to about 0.1% to less than or equal to about 5% of the electrolyte.

7. The battery of claim 1, wherein the inorganic lithium salt comprises lithium hexafluorophosphate.

8. The battery of claim 1, wherein the organic solvent comprises a mixture of ethylene carbonate and ethyl methyl carbonate.

9. The battery of claim 1, wherein the polymeric matrix component comprises, by weight, greater than or equal to about 90% polytetrafluoroethylene, and wherein the polymeric matrix component constitutes, by weight, greater than or equal to about 1% to less than or equal to about 5% of the negative electrode.

10. The battery of claim 1, wherein the electroactive negative electrode particles comprise graphite, silicon, or a combination thereof.

11. The battery of claim 1, wherein the particulate component further comprises an electrically conductive carbon-based material.

12. The battery of claim 1, wherein the polymeric matrix component is substantially free of polyvinylidene fluoride.

13. A battery that cycles lithium ions, the battery comprising:
a positive electrode;
a negative electrode spaced apart from the positive electrode, the negative electrode comprising electroactive negative electrode particles embedded in a polymeric matrix component, the electroactive negative electrode particles comprising graphite particles, silicon particles, or a combination thereof, and the polymeric matrix component comprising polytetrafluoroethylene; and
an electrolyte that provides an ionically conductive pathway for the transport of lithium ions between the positive electrode and the negative electrode, the electrolyte comprising:
an organic solvent;
an inorganic lithium salt; and
a functional additive consisting of a chemical compound having the following formula (1): 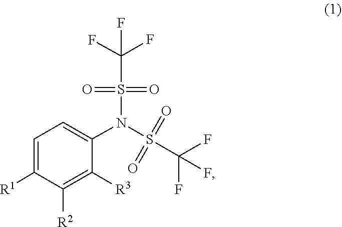
where R¹, R², and R³ are each individually hydrogen, an alkyl group, an alkene group, or a halogen,
wherein, during initial charge of the battery, the functional additive decomposes and forms a solid electrolyte interface on surfaces of the electroactive negative electrode particles that isolates the electroactive negative electrode particles from physical contact with the polymeric matrix component.

14. The battery of claim 13, wherein, during initial charge of the battery, the functional additive decomposes and forms the solid electrolyte interface on surfaces of the electroactive negative electrode particles when the negative electrode is at a potential of greater than or equal to about 1.5 Volts and less than or equal to about 2.2 Volts vs. Li/Li⁺.

15. The battery of claim 13, wherein the electroactive negative electrode particles have a solid electrolyte interface formed on surfaces thereof, and wherein the solid electrolyte interface comprises one or more chemical compounds comprising a bis(trifluoromethanesulfonimide group, trifluoromethyl group, sulfur oxide group, or a combination thereof.

16. The battery of claim 13, wherein the functional additive constitutes, by weight, greater than or equal to about 0.1% to less than or equal to about 5% of the electrolyte.

17. The battery of claim 13, wherein the inorganic lithium salt comprises lithium hexafluorophosphate.

18. The battery of claim 13, wherein the organic solvent comprises a mixture of ethylene carbonate and ethyl methyl carbonate.

19. The battery of claim 13, wherein the polymeric matrix component comprises, by weight, greater than or equal to about 90% polytetrafluoroethylene, and wherein the polymeric matrix component constitutes, by weight, greater than or equal to about 1% to less than or equal to about 5% of the negative electrode.

20. A method of manufacturing a battery comprising a negative electrode and an electrolyte infiltrating the negative electrode, the negative electrode comprising graphite particles embedded in a polytetrafluoroethylene binder, and the electrolyte comprising a chemical compound including a bis(trifluoromethanesulfonimide group and a substituted phenyl group, the method comprising:
applying a constant current at a charge rate of less than or equal to about C/20 to the battery until the battery reaches a predetermined maximum potential; and then
applying a constant voltage to the battery at the predetermined maximum potential until a measured current reaches a charge rate of less than or equal to about C/50.
